(19) United States
(12) Patent Application Publication
(10) Pub. No.: US 2026/0013802 A1
(43) Pub. Date: Jan. 15, 2026

(54) IMAGE GENERATION DEVICE, IMAGE GENERATION SYSTEM, IMAGE GENERATION METHOD AND RECORDING MEDIUM

(71) Applicant: NIHON KOHDEN CORPORATION, Tokyo (JP)

(72) Inventors: Mitsuhiro OURA, Saitama (JP); Kiyoshi TAKEDA, Saitama (JP); Risa KISHI, Saitama (JP); Daisuke HORIGUCHI, Saitama (JP)

(73) Assignee: NIHON KOHDEN CORPORATION, Tokyo (JP)

(21) Appl. No.: 19/257,040

(22) Filed: Jul. 1, 2025

(30) Foreign Application Priority Data
Jul. 9, 2024 (JP) 2024-110440

(51) Int. Cl. A61B 5/00; A61M 16/00; G06T 11/20; G16H 40/63

(52) U.S. Cl. CPC A61B 5/7425; A61B 5/7275; A61M 16/0003; G06T 11/206; G16H 40/63

(57) ABSTRACT

An image generation device according to the presently disclosed subject matter inputs parameter values for a patient monitor from a plurality of medical devices that output the parameter values, and generates a display image of management target-categorized display in which a plurality of the parameter values are collectively arranged by management target functions.

### CROSS-REFERENCE TO RELATED APPLICATIONS

This application is based on and claims priority under 35 USC 119 from Japanese Patent Application No. 2024-110440 filed on Jul. 9, 2024, the contents of which are incorporated herein by reference.

### TECHNICAL FIELD

The presently disclosed subject matter relates to an image generation device, an image generation system, an image generation method, and a recording medium for medical purpose.

### BACKGROUND ART

There are various medical devices for monitoring a patient, such as a patient monitor and a respirator, and a medical worker obtains patient information from these medical devices. A display device that displays patient information is provided for each medical device, and a medical worker comprehensively determines patient information obtained from a plurality of display devices and uses the information for patient management.

In the related art, a plurality of medical devices that monitor a patient each include a display device, and patient information is displayed for each medical device. Therefore, in order to grasp various management target functions of a patient necessary for patient management, advanced medical technology is required. As a result, it may be difficult for a medical worker who is not used to the use of the medical device to grasp a state of the patient.

### SUMMARY

An object of the presently disclosed subject matter is to aggregate patient information and display the patient information in a manner that makes it easy for a medical worker to grasp a state of a patient.

An image generation device according to one embodiment of the presently disclosed subject matter inputs parameter values for a patient monitor from a plurality of medical devices that output the parameter values, and generates a display image of management target-categorized display in which a plurality of the parameter values are collectively arranged by management target functions.

According to the presently disclosed subject matter, the patient information can be aggregated and displayed in a manner that makes it easy for a medical worker to grasp the state of a patient.

### BRIEF DESCRIPTION OF DRAWINGS

FIG. 1 illustrates a status of patient management in an embodiment.

FIG. 2 illustrates a configuration of an image generation device according to the embodiment.

FIG. 3 illustrates a display screen of medical device-categorized display according to Embodiment 1.

FIG. 4 illustrates a display screen of management target-categorized display according to Embodiment 1.

FIG. 5 illustrates a display screen of management target-categorized display according to Embodiment 2.

FIG. 6 illustrates a management target-categorized image of a ventilation function according to Embodiment 2.

FIG. 7 illustrates a display screen of management target-categorized display according to Embodiment 3.

FIG. 8 illustrates a display screen of management target-categorized display according to Embodiment 4.

FIG. 9 illustrates a display screen of management target-categorized display according to a modification of Embodiment 4.

### DESCRIPTION OF EMBODIMENTS

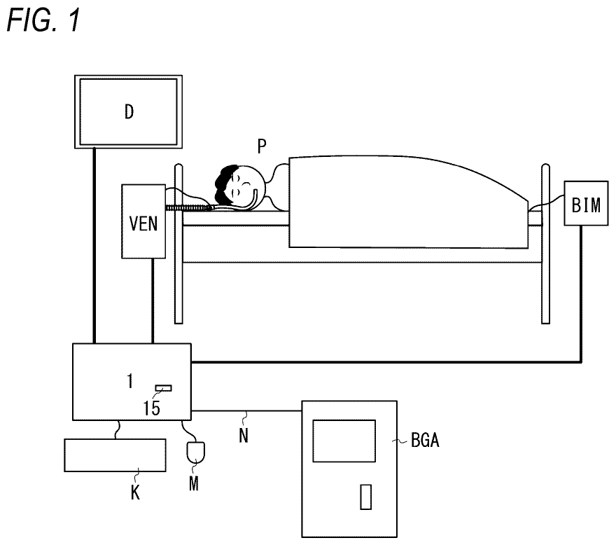

FIG. 1 illustrates a status of patient management in embodiments. A respirator VEN, which is a medical device ME, and a patient monitor BIM are attached to a patient P. An image generation system according to the embodiments can include a plurality of medical devices ME such as the respirator VEN and the patient monitor BIM, a display device D, and an image generation device 1. The respirator VEN and the patient monitor BIM obtain parameter values of various parameters for the patient monitor by measuring a state of the patient P, and transmit the parameter values to the image generation device 1. The image generation device 1 stores the received parameter values. A mouse M and a keyboard K as input devices are connected to the image generation device 1, and a medical worker can operate the image generation device 1. Further, blood drawn from the patient P is measured by a blood gas analyzer BGA which is the medical device ME. The parameter values for the patient monitor obtained by the measurement are input from the blood gas analyzer BGA to the image generation device 1 via an in-hospital network N.

The parameter values of the blood gas analyzer BGA can also be input to the image generation device 1 via a removable storage such as a USB. In addition, these parameter values can be recorded by printing, registration, or the like, and can be manually input using the mouse M and the keyboard K while viewing the record.

The image generation device 1 generates a display image from the parameter values input from the plurality of medical devices ME and transmits the display image to the display device D. The blood collected from the patient P is put into the blood gas analyzer BGA, and in the present embodiment, the measured parameter values are updated and displayed on the display device D at a timing when the parameter values are input to the image generation device 1 via the in-hospital network N. The parameter values of the respirator VEN and the patient monitor BIM are continuously updated and displayed on the display device D.

In the embodiment illustrated in FIG. 1, the display device D is installed on a bedside, but the display device D may be displayed on a display at a place other than the bedside in the hospital, or a tablet or the like carried by a medical worker may be used as the display device D. The respirator VEN, the patient monitor BIM, and the image generation device 1 can be partially or entirely arranged below a bed or the like. The image generation device 1 may be provided in another room. The mouse M and the keyboard K can be removed when unnecessary.

The patient monitor BIM performs measurement and calculation using a sensor or the like attached to the patient P. Then, as the parameter values, the transcutaneous oxygen saturation SpO2, an end-tidal carbon dioxide partial pressure etCO2, a vital sign BP, a body temperature TEMP, a respiration rate RR, and the like are transmitted. The transmitted parameter values are input to the image generation device 1.

In the respirator VEN, respirator is performed with the set contents, and measurement and calculation are also performed. Then, as the parameter values, fraction of inspired oxygen FiO2, a positive end-expiratory pressure PEEP, a flow velocity FR, a tidal volume VT, a plateau pressure PPLAT, a driving pressure ΔP, mechanical power MP, an airway resistance AR, compliance CP, the respiration rate RR, and the like are transmitted. The transmitted parameter values are input to the image generation device 1.

In the blood gas analyzer BGA, the collected blood is set in the analyzer and measured for calculation. Then, as parameter values, arterial blood transcutaneous oxygen saturation SaO2, an arterial blood oxygen partial pressure PaO2, arterial blood carbon dioxide partial pressure PaCO2, pondus hydrogenii pH, and the like are obtained. These parameter values are input to the image generation device 1 via the in-hospital network N connected to the blood gas analyzer BGA.

In the image generation device 1, the value of the respiration rate RR is obtained from both the patient monitor BIM and the respirator VEN. The image generation device 1 calculates a value of the arterial blood oxygen partial pressure PaO2 obtained by the blood gas analyzer BGA and a value of fraction of inspired oxygen FiO2 obtained by the respirator VEN to calculate a value of P/F (PF).

The parameter value is a concept including setting information (for example, PEEP, FiO2, the tidal volume VT, and the like of the respirator VEN) of the medical device ME capable of estimating physiological information on the patient P in addition to a value of the physiological information obtained from a body of the patient P (for example, a blood pressure value, the respiration rate, the body temperature, a pulse rate, and the like obtained from various sensors). The setting information is basically changed by a setting operation of the medical worker, but it may be automatically changed by the respirator VEN according to the patient state (for example, the value of the tidal volume VT is dynamically changed).

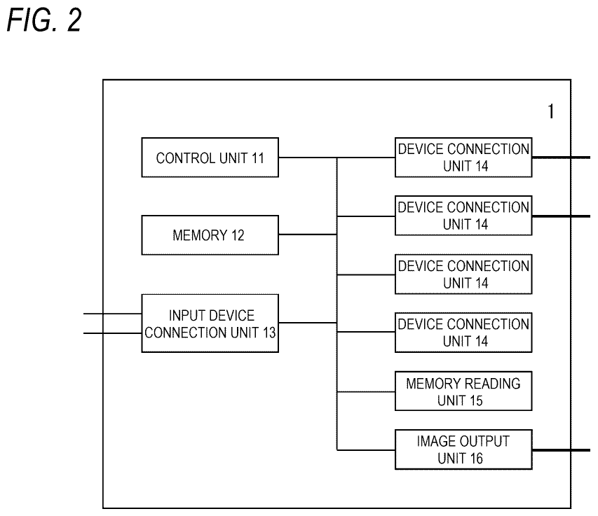

FIG. 2 illustrates a configuration of the image generation device 1 according to the embodiment. The image generation device 1 can include a control unit 11 that is a CPU, one or more memories 12, an input device connection unit 13, a plurality of device connection units 14, a memory reading unit 15, and an image output unit 16, which are connected by a bus. The memory 12 stores a computer program for causing the image generation device 1, which is a computer, to execute various procedures. The computer program can be stored in the memory 12 via a computer-readable recording medium. The mouse M and the keyboard K, which are input devices, are connected to the input device connection unit 13. The patient monitor BIM and the respirator VEN are connected to each of the plurality of device connection units 14, and various parameter values are captured. Another device can be connected to the device connection unit 14 that is not connected. A removable storage can be connected to the memory reading unit 15 to read settings, software, and the like. The display device D is connected to the image output unit 16 and transmits a display image.

In the memory 12, a tag is added to each parameter and a parameter value is stored. As the tag, a medical device-categorized tag and a management target-categorized tag are used. The medical device-categorized tag is a tag indicating the obtained medical device ME, and in the embodiment, tags of “BIM”, “VEN”, and “BGA” are assigned to parameters. The management target-categorized tag is a tag indicating a management target function, and one or more tags of an “oxygenation function”, a “ventilation function”, “work of breathing”, “lung mechanics”, and “others” are assigned to the parameters. The control unit 11 generates a display image using the medical device-categorized tag or the management target-categorized tag.

### Embodiment 1

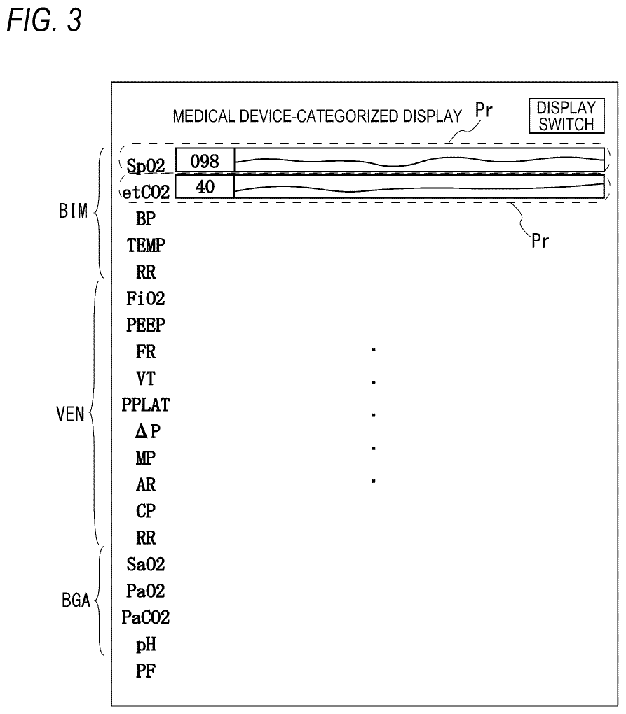

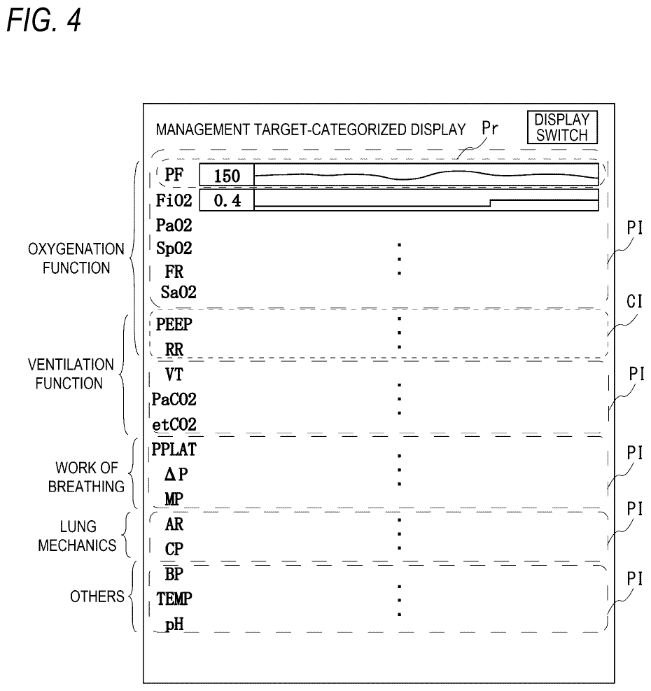

In Embodiment 1, the display image of the medical device-categorized display illustrated in FIG. 3 and the display image of the management target-categorized display illustrated in FIG. 4 can be generated by the image generation device 1, and switched and displayed on the display device D. By using the mouse M, which is an input device, to point to a “display switch” button in the upper right corner of the display image and click, whereby the display is switched between the medical device-categorized display and the management target-categorized display.

FIG. 3 illustrates a display screen of the medical device-categorized display according to Embodiment 1. In the display screen of FIG. 3, the display image generated by the image generation device 1 illustrated in FIGS. 1 and 2 is displayed on the display device D. In the medical device-categorized display, regarding the parameter values, the parameter values at the current time point collectively arranged and displayed in a list for each obtained medical device are numerically displayed, and the parameter values up to the current time point being displayed are trend-displayed by line graphs or point graphs. The examples of SpO2 and etCO2 illustrated in FIG. 3 are line graphs. In the medical device-categorized display according to Embodiment 1, the image generation device 1 generates a display image in which a plurality of individual parameter images Pr are collectively arranged and displayed for respective medical devices using the medical device-categorized tags and a plurality of medical device-categorized images are simultaneously displayed, and displays the display image on the display device D.

In FIG. 3, the individual parameter image Pr displayed for one parameter of SpO2 is surrounded by a dotted line in an upper portion. The individual parameter image Pr is horizontally long, and display contents are a parameter name, a parameter value at the current time point, and a trend display using a line graph or the like from a left side. The parameter value and the trend display are described in a frame. “098” illustrated in FIG. 3 indicates that the parameter value of the SpO2 at the current time point is 98%. Below the individual parameter image Pr of SpO2, the individual parameter image Pr of etCO2 is illustrated. For etCO2, the parameter value at the current time point is 40 mmHg. The individual parameter images Pr related to other parameters including BP and parameters below BP are also horizontally long, and the display content is similarly displayed in each individual parameter image Pr. In FIG. 3, description of the parameter values and the trend display is omitted for the individual parameter images Pr related to other parameters including BP and parameters below BP. As described above, in the display image of the medical device-categorized display according to Embodiment 1, the plurality of individual parameter images Pr are arranged in a vertical direction and displayed in a list.

In the medical device-categorized display illustrated in FIG. 3, the individual parameter images Pr from SpO2 to RR are a medical device-categorized image in which parameters obtained from the patient monitor BIM are displayed and parameter values and the like of the patient monitor BIM are collectively arranged. The individual parameter images Pr from FiO2 to RR are a medical device-categorized image in which parameters obtained from the respirator VEN are displayed and parameter values and the like of the respirator VEN are collectively arranged. The individual parameter images Pr from SaO2 to pH are a medical device-categorized image in which parameters obtained from the blood gas analyzer BGA are displayed and parameter values and the like of the blood gas analyzer BGA are collectively arranged. In the medical device-categorized display illustrated in FIG. 3, a plurality of medical device-categorized images in which a plurality of individual parameter images Pr are collectively arranged for medical devices are displayed side by side. The parameter value of PF, which is a parameter illustrated at the bottom, is calculated by the image generation device 1 from the value of the arterial blood oxygen partial pressure PaO2 obtained in the blood gas analyzer BGA and the value of the fraction of inspired oxygen FiO2 obtained in the respirator VEN.

FIG. 4 illustrates a display screen of management target-categorized display according to Embodiment 1. In the display screen of FIG. 4, a display image generated by the image generation device 1 illustrated in FIGS. 1 and 2 is displayed on the display device D. In the management target-categorized display according to Embodiment 1, parameter values obtained from the medical devices ME are collectively arranged and displayed in a list by the management target functions of the patient P. As for each parameter, the parameter value at the current time point being displayed is numerically displayed, and the parameter value up to the current time point being displayed is trend-displayed by a line graph, a point graph, or the like. The examples of PF and FiO2 illustrated in FIG. 4 are line graphs. In the management target-categorized display according to Embodiment 1, the image generation device 1 uses management target-categorized tags to generate a plurality of management target-categorized images in which the plurality of individual parameter images Pr are collectively arranged and displayed for respective management targets, and generates and displays a display image in which the plurality of management target-categorized images are simultaneously displayed. In FIG. 4, each management target-categorized image is a collection of images of a plurality of individual parameter areas.

Similarly to FIG. 3, the individual parameter image Pr according to the embodiment illustrated in FIG. 4 is horizontally long, and the display contents are a parameter name, a parameter value at the current time point being displayed, a trend display using a line graph or the like from a left side. The plurality of individual parameter images Pr are arranged in the vertical direction and generated in a list. The management target-categorized display is basically display in which the plurality of individual parameter images Pr in the medical device-categorized display are collectively arranged and displayed by the management target.

In the management target-categorized display of FIG. 4, “PF” described at a left end of the upper portion is the parameter name, and “150” on a right side thereof is the parameter value at the current time point. A line graph or the like on a right side of the parameter value is a trend display up to the current time point. The parameter value and the trend display are described in a frame. The parameter value of PF is calculated by the image generation device 1 from the value of the arterial blood oxygen partial pressure PaO2 obtained by the blood gas analyzer BGA and the value of the fraction of inspired oxygen FiO2 obtained by the respirator VEN. In FiO2, the parameter value at the current time point is 0.4. The parameter values and the trend display for parameters including FiO2 and parameters below FiO2 are omitted.

In FIG. 4, PF to RR are displayed as a management target-categorized image of the “oxygenation function” in which the individual parameter images Pr of the oxygenation function are collectively arranged, including right areas of these parameter names. In addition, PEEP to etCO2 are displayed as a management target-categorized image of the “ventilation function” in which the individual parameter images Pr of the ventilation function are collectively arranged, including right areas of these parameter names. PPLAT to MP below are displayed as a management target-categorized image of the “work of breathing” in which the individual parameter image Pr areas of the work of breathing are collectively arranged, including right areas of these parameter names. Further, AR to CP are displayed as a management target-categorized image of “lung mechanics” in which the individual parameter images Pr of lung mechanics are collectively arranged, including right areas of these parameter names. BP to pH are displayed as a management target-categorized image of “others” in which the individual parameter images Pr areas of others are collectively arranged, including right areas of these parameter names.

PEEP and RR are parameters common to the oxygenation function and the ventilation function, and are included in two management target-categorized images of the “oxygenation function” and the “ventilation function”. Further, the individual parameter images Pr included in the plurality of management target-categorized images are collectively arranged and displayed as a common management target image CI as indicated by a dotted line frame. Since the PEEP and the RR are provided with two management target-categorized tags of the “oxygenation function” and the “ventilation function”, they are collectively displayed as the common management target image CI. In Embodiment 1, the RR displays an average value of the parameter values of the RR obtained from the patient monitor BIM and the respirator VEN.

The individual parameter images Pr from PF to SaO2 in the management target-categorized image of the “oxygenation function” are parameters of only the oxygenation function, and are collectively arranged and displayed as a specific management target image PI as indicated by a dotted line frame. Further, the individual parameter images Pr from VT to etCO2 in the management target-categorized image of the “ventilation function” are parameters of only the ventilation function, and are collectively arranged and displayed as the specific management target image PI as indicated by a dotted line frame. The individual parameter images Pr not included in the plurality of management target-categorized images are collectively arranged and displayed as the specific management target image PI. In the example of FIG. 4, the common management target image CI common to the “oxygenation function” and the “ventilation function” is displayed between the specific management target image PI of only the “oxygenation function” and the specific management target image PI of only the “ventilation function”.

In the embodiment of FIG. 4, the management target-categorized image of “work of breathing” from PPLAT to MP is not included in the other management target-categorized images, and is the specific management target image PI of “work of breathing”. Similarly, AR and CP are specific management target images PI of “lung mechanics”. Further, the management target-categorized image from BP to pH of “others” is collectively arranged and displayed as the specific management target image PI. By collectively arranging these images by the management target functions and collectively displaying the management target-categorized images, the medical worker who views the management target-categorized display illustrated in FIG. 4 can easily grasp the status of the patient P for each management target function.

As described above, the management target-categorized image of the “oxygenation function” includes the specific management target image PI of the “oxygenation function” that displays a parameter value not common to other management target-categorized images, and the common management target image CI that displays a parameter value also included in the management target-categorized image of the “ventilation function”. The management target-categorized image of the “ventilation function” includes the specific management target image PI of the “ventilation function” that displays a parameter value not included in other management target-categorized images, and the common management target image CI that displays a parameter value also included in the management target-categorized image of the “oxygenation function”. Then, the common management target image CI is displayed between the specific management target image PI of the “oxygenation function” and the specific management target image PI of the “ventilation function”. Accordingly, the medical worker who views the management target-categorized display can easily grasp the status of the patient P for each management target function.

In the management target-categorized display of FIG. 4, it is easy to grasp the status of the patient P for each management target function. However, some medical workers may be accustomed to the medical device-categorized display illustrated in FIG. 3. Therefore, in Embodiment 1, by moving a cursor with the mouse M and clicking “display switch” on the upper right of the display image, it is possible to switch and display the medical device-categorized display illustrated in FIG. 3 and the management target-categorized display illustrated in FIG. 4.

The memory 12 of the image generation device 1 stores a computer program for causing a computer to execute an input procedure of inputting a plurality of parameter values for a patient monitor from a plurality of medical devices ME, and a screen generation procedure of generating a display image for the management target-categorized display in which the parameter values are collectively arranged by the management targets. The computer program can be recorded on a computer-readable recording medium. In addition, an image generation method is performed in which parameter values are input from a plurality of medical devices that output parameter values for a patient monitor, and a display image of management target-categorized display in which the parameter values are collectively arranged for the management target functions is generated.

### Modification

In Embodiment 1, the management target-categorized display in FIG. 4 and the medical device-categorized display in FIG. 3 can be switched and displayed. However, only the management target-categorized display as illustrated in FIG. 4 may be displayed. Further, in Embodiment 1, in the management target-categorized display illustrated in FIG. 4, a boundary between the management target-categorized images is not displayed. However, the boundary between the management target-categorized images may be displayed by displaying a frame, a colored background, or the like. Further, which area corresponds to which management target function may be displayed by character notation or the like. Also in the medical device-categorized display in FIG. 3, a boundary for each medical device may be displayed or characters may be written. Further, the management target-categorized display may be made with the management target-categorized image of only the specific management target images PI without the common management target images CI.

### Embodiment 2

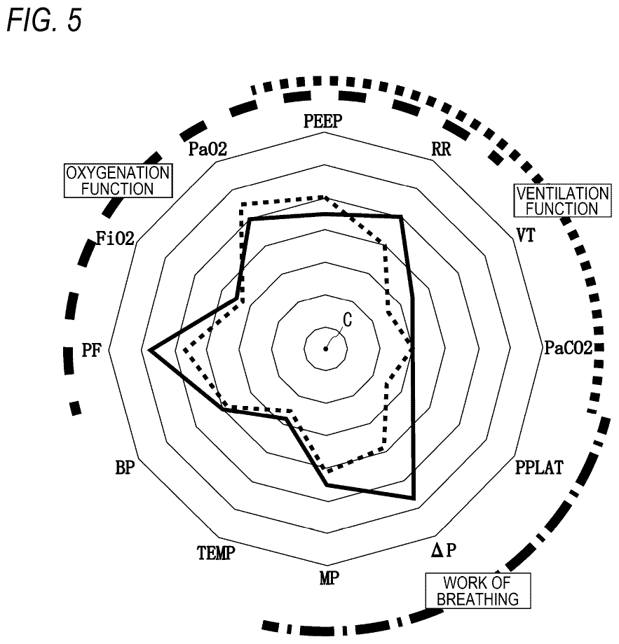

FIG. 5 illustrates a display screen of management target-categorized display according to Embodiment 2. In the display screen of FIG. 5, the display image generated by the image generation device 1 illustrated in FIGS. 1 and 2 is displayed on the display device D. In the management target-categorized display according to Embodiment 2, the parameter values obtained from the medical device are displayed by a radar chart collectively arranged by the management target functions of the patient P. In Embodiment 2, the display image is formed by omitting a part of the display of the parameter values transmitted from the medical device ME.

In the radar chart display of FIG. 5 in Embodiment 2, a plurality of regular dodecagons having different sizes with a center C in common are illustrated. Then, 12 parameter names are displayed annularly outside the regular dodecagon, and in the regular dodecagon display area, the parameter values of the respective parameters are connected and displayed in a radar chart. A thick solid line on the radar chart indicates the parameter values at the current time point being displayed, and a dotted line indicates the average parameter value in a predetermined time until the current time point being displayed.

The five parameters from the PF positioned on the left to the RR toward the upper right indicate that the management target function is the “oxygenation function”. The four parameters from the uppermost PEEP to PaCO2 toward the right side indicate that the management target function is the “ventilation function”. Further, from PPLAT to the lowermost MP, the management target function is the “work of breathing”. An arc-shaped dotted line illustrated on an outer periphery indicates a range of the management target function, and management target function names indicating the management target function are displayed superimposed on the arc.

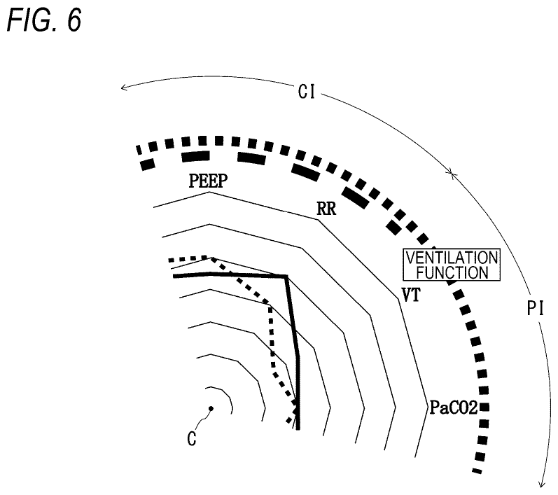

In Embodiment 2, the management target-categorized image, the specific management target image PI, and the common management target image CI are fan-shaped, and a display image is formed by combining these images. The individual parameter image Pr also has a fan shape. FIG. 6 illustrates a management target-categorized image of the ventilation function according to Embodiment 2. The management target-categorized image has a fan shape spreading out from the center C. The specific management target image PI of the ventilation function and the common management target image CI common to the oxygenation function are also fan-shaped. Also in management target-categorized display according to Embodiment 2, a plurality of management target-categorized images for displaying parameter values by the management targets are generated by using the management target-categorized tags assigned to the parameters, and a display image for simultaneously displaying the plurality of management target-categorized images is generated and displayed. Since Embodiment 2 is the radar chart display, the fan-shaped display of the management target-categorized image or the like is displayed with a line so that positions of the parameter values are connected to the fan-shaped display of the adjacent management target-categorized image or the like with a straight line.

In FIG. 5, a fan-shaped area indicated by a thick long-dotted arc from PF to RR is a management target-categorized image of the “oxygenation function”, and a fan-shaped area indicated by a thick short-dotted arc from PEEP to PaCO2 is a management target-categorized image of the “ventilation function”. A fan-shaped area indicated by the thick alternate long and short dash line arc is a management target-categorized image of the “work of breathing”. Further, the fan-shaped area from PF to PaO2 is the specific management target image PI of the “oxygenation function”, and the fan-shaped area from PEEP to RR is the common management target image CI of the “oxygenation function” and the “ventilation function”. The medical worker who views the management target-categorized display of the radar chart display illustrated in FIG. 5 can easily grasp the status of the patient P for each management target function.

### Modification

The management target-categorized images, the specific management target image PI, and the common management target image CI having fan shapes according to Embodiment 2 are displayed so that the areas are identified by arc-shaped dotted lines or the like on the outer periphery. However, the above images may be displayed so that the areas are identified by another display method such as a color area, a display color of a character, or a display color of a line. In addition, the solid lines and the dotted lines of Embodiment 2 connect and display the parameter values at the current time point being displayed and the average parameter value in the predetermined time until the current time point being displayed. However, another parameter value such as a parameter value before the predetermined time may be connected and displayed in the radar chart. Further, the management target-categorized display may be made with the management target-categorized image of only the specific management target images PI without the common management target images CI. In addition, each parameter value of the radar chart display may be normalized and displayed according to a normal range.

### Embodiment 3

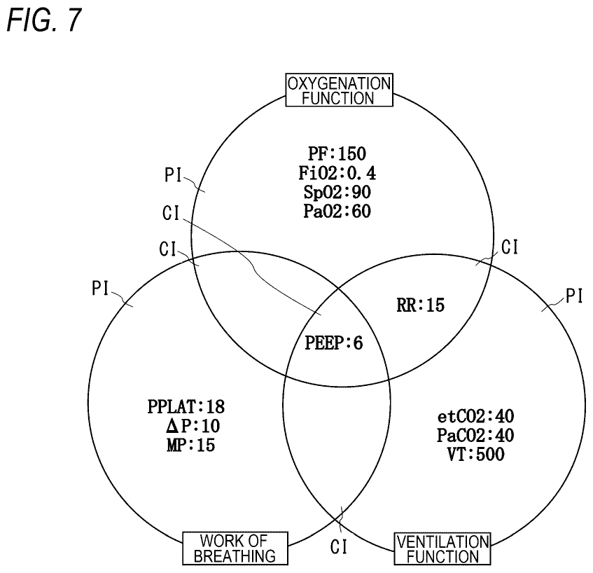

FIG. 7 illustrates a display screen of management target-categorized display according to Embodiment 3. In the display screen of FIG. 7, the display image generated by the image generation device 1 illustrated in FIGS. 1 and 2 is displayed on the display device D. In the management target-categorized display according to Embodiment 3, the parameter values obtained from the medical device ME are collectively arranged by the management target functions of the patient P and displayed in a Venn diagram. In Embodiment 3, the display image is formed by omitting a part of the parameter values transmitted from the medical device ME.

In the display of the Venn diagram of FIG. 7 in Embodiment 3, the inside of each circle is the management target-categorized image. An area that does not overlap another circle in the management target-categorized image is the specific management target image PI, and an area that overlaps another circle is the common management target image CI. The display image of the management target-categorized display is represented by a Venn diagram with a plurality of closed curves, the parameter values having the same management target function are displayed inside the corresponding closed curve, and a plurality of parameter values are displayed in the Venn diagram. In each circle, a management target function name indicating a management target function is displayed.

In the specific management target image PI of the “oxygenation function”, regarding the parameters of PF, FiO2, SpO2, and PaO2, the parameter values are indicated by numerical values. Further, RR is displayed in the common management target image CI of the “oxygenation function” and the “ventilation function”, and PEEP is displayed in the common management target image CI of the “oxygenation function”, the “ventilation function”, and the “work of breathing”. Further, etCO2, PaCO2, and VT are displayed in the specific management target image PI of the “ventilation function”, and PPLAT, AP, and MP are displayed in the specific management target image PI of the “work of breathing”. Also in the management target-categorized display according to Embodiment 3, a plurality of management target-categorized images for displaying parameter values by the management targets are generated by using the management target-categorized tags assigned to the parameters, and a display image for simultaneously displaying a plurality of management target-categorized images is generated and displayed.

In Embodiment 3, the parameter names and the parameter values are displayed inside the closed curve, but the parameter values or the like having the same management target function may be displayed in the Venn diagram by associating the parameter values or the like with the inside of the corresponding closed curve. For example, the parameter value or the like may be displayed protruding from the inside of the closed curve, or a line may be drawn from the inside of the closed curve to the outside, and the parameter value or the like may be displayed at an end of the drawn line.

### Embodiment 4

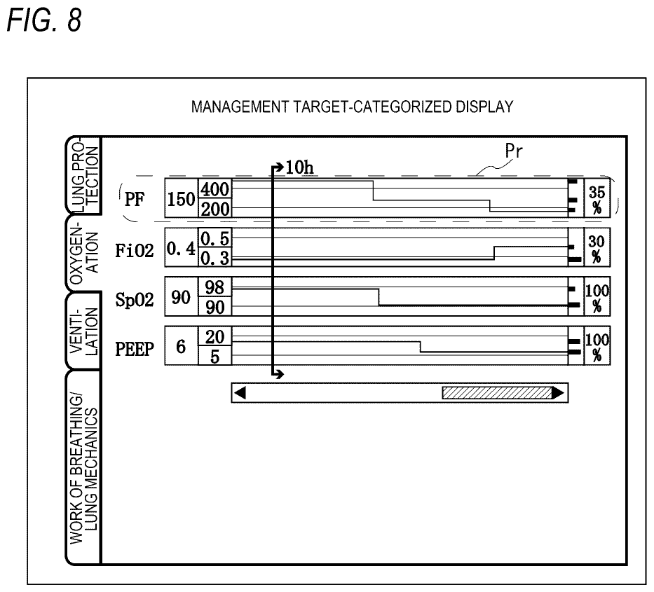

FIG. 8 illustrates a display screen of management target-categorized display according to Embodiment 4. In the display screen of FIG. 8, the display image generated by the image generation device 1 illustrated in FIGS. 1 and 2 is displayed on the display device D. In the management target-categorized display according to Embodiment 4, the parameter values obtained from the medical device ME are collectively arranged and displayed in a list by the management target functions of the patient P. As for each parameter, the parameter values at the current time point being displayed are numerically displayed, and the parameter values up to the current time point being displayed are trend-displayed by line graphs, point graphs, or the like. Also in Embodiment 4, the image generation device 1 generates the display image of the management target-categorized display using the management target-categorized tags assigned to the parameters.

As illustrated in FIG. 8, the individual parameter image Pr according to the embodiment is horizontally long as in Embodiment 1. From the left side, the parameter name, the parameter value at the current time point being displayed, an upper limit value and a lower limit value of an appropriate range, the trend display by the line graphs or the like, and a histogram of the parameter value during a predetermined period are displayed, and an appropriate range ratio during the predetermined period is displayed as a percentage.

The upper limit value and the lower limit value of the appropriate range are indicated by numerical values, and in the trend display, the appropriate range is indicated by two horizontal straight lines provided in an upper portion and a lower portion. A vertical line having an arrow at an end portion illustrated in the trend display is a predetermined period setting bar indicating a predetermined period, and a right side of the vertical line is the predetermined period. The predetermined period can be changed by moving the predetermined period setting bar to the left or right using the mouse M. The length of the predetermined period can be confirmed by a numerical value displayed at the end of the arrow of the predetermined period setting bar. In FIG. 8, the predetermined period is set to 10 hours. The predetermined period may be a fixed setting value, or may be a setting value directly input from the keyboard K. In this case, the predetermined period setting bar or the like may not be displayed.

The histogram displayed on the right side of the trend display indicates a distribution of parameter values during the predetermined period. In addition, the appropriate range ratio on the right side indicates a percentage of time when the parameter value is in the appropriate range during the predetermined period. Horizontal straight lines and the histogram representing the upper limit value and the lower limit value of the appropriate range are display indicating to what extent the appropriate range is adapted, and are appropriate range conformity display. Further, the appropriate range ratio is also the appropriate range conformity display.

In FIG. 8, PF indicates that the parameter value is within the appropriate range in 90% of the predetermined period. Further, FiO2 and the like indicate that the parameter value is within the appropriate range in 100% of the predetermined period. A display range of the trend display can be moved to the left and right by a lower scroll bar or the like at the lower portion.

The appropriate range conformity display may be not only the histogram and the appropriate range ratio % illustrated in FIG. 8 but also other display. For example, during the predetermined period, the appropriate range conformity display may be a time during which the parameter value is within the appropriate range, a time (deviation period) during which the parameter value is not within the appropriate range, or the like. In addition, instead of the histogram, a box plot or the like may be shown together with a horizontal straight line or the like between the upper limit value and the lower limit value of the appropriate range to indicate the appropriate range conformity display. In addition to this, the appropriate range conformity display may be expressed by another statistical notation.

In FIG. 8, a tab for oxygenation positioned on the left side is selected, and a sheet of a management target-categorized image indicating the oxygenation function of the patient P is displayed. In addition to the tab for oxygenation, tabs for lung protection, ventilation, and work of breathing/lung mechanics can be selected. As a result, it is possible to switch sheets illustrating the management target-categorized images of a lung protection function, the ventilation function, and the work of breathing/lung mechanics of the patient P. The sheets are switched by placing the cursor on the tab and clicking the mouse M.

In the management target-categorized display according to Embodiment 4, a plurality of management target-categorized image for displaying parameter values by the management targets are generated, and the plurality of management target-categorized images are switched and displayed. Therefore, when there is a common management target image CI, the common management target image CI is displayed on a plurality of sheets.

### Modification

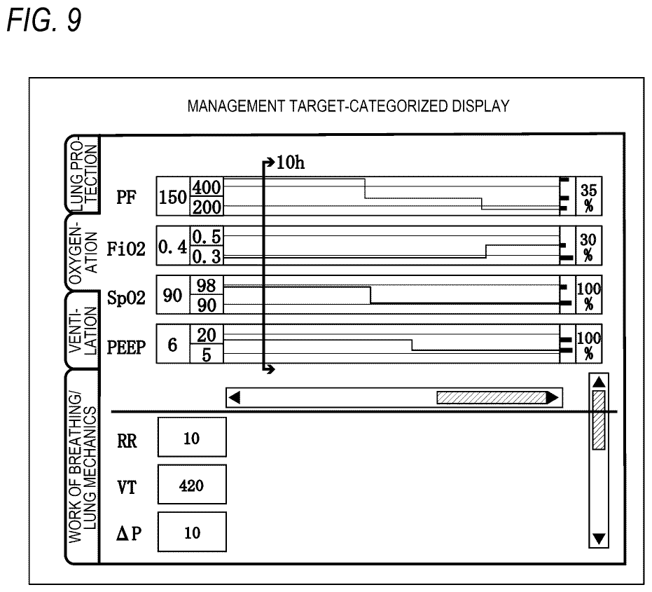

FIG. 9 illustrates a display screen of management target-categorized display according to a modification of Embodiment 4. In the display screen of FIG. 9, the display image generated by the image generation device 1 illustrated in FIGS. 1 and 2 is displayed on the display device D. Also in the management target-categorized display according to the modification, the parameter values obtained from the medical device ME are collectively arranged and displayed in a list by the management target functions of the patient P. As for each parameter, the parameter values at the current time point being displayed are numerically displayed, and the parameter values up to the current time point being displayed are trend-displayed by line graphs, point graphs, or the like.

In the display screen according to the modification, the sheet of the oxygenation function is displayed by selecting the tab. PF to PEEP in an upper portion of the sheet are parameters of the oxygenation function and are displayed in detailed display. In the detailed display, similarly to Embodiment 4, for each parameter, the parameter value at the current time point being displayed is numerically displayed, and is trend-displayed. In addition, the appropriate range display, the histogram display, and the appropriate range conformity display are also performed, and a predetermined period setting bar is also displayed. On the other hand, RR to ΔP in the lower portion are displayed by simple display. In the simple display, only the parameter values at the current time point being displayed are displayed. The parameters displayed in the lower portion are parameters other than parameters of the oxygenation function, and a vertical scroll bar displayed on the right side can move up and down to display hidden parameters.

Also in the modification, a plurality of management target-categorized images are switched and displayed. However, in the management target-categorized display according to the modification, the selected management target function is displayed in detail, and the unselected management target functions are simply displayed. The simple display displays a part of the detailed display, and the detailed display and the simple display are not limited to those described in FIG. 9.

In Embodiment 4 and the modification, the management target-categorized image to be displayed is switched by selecting a tab. However, this is an example of switching, and the management target-categorized images may be switched by another method, such as displaying “change management target” in the display image and sequentially switching the management target-categorized images by clicking the display, or displaying a selection display of the management target-categorized images by right-clicking the mouse M and switching the management target-categorized images by selection.

In the image generation system according to the above embodiments, the information of all the medical devices ME is displayed on one display device D. However, when the information on the plurality of medical devices ME is aggregated and displayed on at least one display device D, the information may be displayed on the plurality of display devices. The image generation device 1 and the display device D may be integrally formed as a device of the same housing. Further, an image generation function of the image generation device 1 may be provided in the BIM or the VEN, and the above image may be displayed using a display of the BIM or the VEN as the display device D. At this time, the BIM and the VEN may also be integrally formed.

In addition, the specific configurations are not limited to the embodiments, and modifications and the like of the design without departing from the gist of the presently disclosed subject matter are also included in the presently disclosed subject matter. In addition, as long as there is no particular contradiction or problem in the purpose, configuration or the like of the above embodiments and modifications, techniques of the above embodiments and modifications can be used and combined.

1. An image generation device, wherein
parameter values for a patient monitor are input from a plurality of medical devices that output the parameter values, and
a display image of management target-categorized display in which a plurality of the parameter values are collectively arranged by management target functions is generated.

2. The image generation device according to claim 1, wherein
the management target function includes an oxygenation function and a ventilation function.

3. The image generation device according to claim 2, wherein
the management target function further includes at least one of work of breathing and lung mechanics,
the medical device includes a patient monitor and a respirator, and
the parameter value includes at least three of transcutaneous oxygen saturation SpO2, an end-tidal carbon dioxide partial pressure etCO2, fraction of inspired oxygen FiO2, a tidal volume VT, an airway resistance AR, and compliance CP.

4. The image generation device according to claim 1, wherein
a display image of medical device-categorized display in which the plurality of parameter values are collectively arranged by the medical devices and the display image of the management target-categorized display is able to be generated by switching.

5. The image generation device according to claim 1, wherein
a display image of management target-categorized display that simultaneously displays a plurality of the management target-categorized images of the management target functions is generated.

6. The image generation device according to claim 5, wherein
at least two of the plurality of management target-categorized images include a specific management target image that displays the parameter value not included in another management target function and a common management target image that displays the parameter value also included in the other management target function, and
the common management target image is positioned between the two specific management target images.

7. The image generation device according to claim 6, wherein
the display image of the management target-categorized display is a radar chart display of the plurality of parameter values.

8. The image generation device according to claim 6, wherein
the display image of the management target-categorized display is represented by a Venn diagram including a plurality of closed curves indicating the management target-categorized images, and
the plurality of parameter values are displayed in the Venn diagram by associating the parameter values having a same management target function with inside the corresponding closed curve and displaying the parameter values inside the corresponding closed curve.

9. The image generation device according to claim 1, wherein
the display image is generated as the management target-categorized image for each management object, and
the plurality of management target-categorized images are switched and displayed.

10. The image generation device according to claim 9, wherein
the management target-categorized image displays the parameter value which is a management target in detail, and simply displays the parameter value which is not the management target.

11. The image generation device according to claim 1, wherein
the management target-categorized display is a trend display of the plurality of parameter values.

12. The image generation device according to claim 1, wherein
appropriate range display or appropriate range conformity display is provided together with the parameter value.

13. An image generation system comprising:
a plurality of medical devices configured to output parameter values for a patient monitor;
a display device; and
an image generation device configured to input a plurality of the parameter values from the medical device, wherein
the image generation device generates a display image of management target-categorized display that displays the plurality of parameter values for management targets, and outputs the display image to the display device.

14. An image generation method comprising:
inputting parameter values for a patient monitor from a plurality of medical devices that output the parameter values; and
generating a display image of management target-categorized display in which the parameter values are collectively arranged by management target functions.

15. A computer-readable recording medium having a computer program recorded thereon, the computer program causing a computer to execute:
inputting parameter values for a patient monitor from a plurality of medical devices that output the parameter values; and
generating a display image for management target-categorized display in which the parameter values are collectively arranged by management targets.
